(19) United States
(12) Patent Application Publication
(10) Pub. No.: US 2008/0108099 A1
(43) Pub. Date: May 8, 2008

(54) METHODS FOR DIAGNOSING CHRONIC DIARRHEA

(76) Inventor: Thomas M. Donndelinger, Nampa, ID (US)

Correspondence Address: KIRTON AND MCCONKIE, 60 EAST SOUTH TEMPLE,, SUITE 1800, SALT LAKE CITY, UT, 84111, US

(21) Appl. No.: 11/876,590

(22) Filed: Oct. 22, 2007

Related U.S. Application Data
(63) Continuation-in-part of application No. 11/613,717, filed on Dec. 20, 2006.
(60) Provisional application No. 60/752,618, filed on Dec. 21, 2005.

(51) Int. Cl. C12Q 1/40; C12Q 1/34; C12Q 1/44; C12Q 1/37

(52) U.S. Cl. 435/19; 435/18; 435/22; 435/24

(57) ABSTRACT

Methods for diagnosing chronic diarrhea and other gastrointestinal conditions are disclosed. In the methods, a sample of gastrointestinal secretions may be obtained from a control group; or a group of people who have been diagnosed with either healthy gastrointestinal tracts or a gastrointestinal condition, like chronic diarrhea. The samples from the control group may be analyzed in any manner to determine the levels of secretions, like antimicrobial proteins, mitochondria, and digestive enzymes, in gastrointestinal secretions. The results of the sample analysis may be used to create a database containing profiles of normal and abnormal gastrointestinal secretions. As the database is created and specific secretion level abnormalities are identified, patients may be diagnosed with these abnormalities and be treated by adjusting the levels of specific secretions. Furthermore, gastrointestinal samples from subsequent patients may be analyzed and compared with the database in order to determine which secretion levels, if any, are abnormal.

### CROSS-REFERENCE TO RELATED APPLICATION

This is a continuation-in-part application of U.S. Utility patent application Ser. No. 11/613,717, filed Dec. 20, 2006, which claims priority to U.S. Provisional Patent Application 60/752,618, filed Dec. 21, 2005, the entire disclosures of which are both hereby incorporated by reference.

### BACKGROUND

Many people suffer from the medical condition of chronic diarrhea, or diarrhea that lasts for more than two weeks. Although persistent, this problem is usually painless and is generally not accompanied by more serious conditions, such as bleeding, anemia, weight loss, or fatigue. Nevertheless, this condition can be a nuisance and an embarrassment to many people. Accordingly, doctors and researchers have long searched for causes of the ailment as well as its treatments.

There are many different causes of chronic diarrhea. For example, some types of chronic diarrhea may be caused by infection, which, in turn, may have been caused by parasites, bacteria, viruses, or the like. In other cases, chronic diarrhea may be caused by medications, antibiotics, food additives, or drugs ingested into the body. In yet other cases, chronic diarrhea may be caused by more serious medical conditions, such as cancer, pre-malignant and malignant lesions, tumors, diabetes, thyroid and other endocrine diseases, food allergies, reduced blood flow to the intestine, colitis, and/or Crohn's disease. Previous surgery or radiation treatments on the abdomen or gastrointestinal tract may also be the source of some forms of chronic diarrhea.

Yet, despite the knowledge regarding chronic diarrhea, there are some cases of chronic diarrhea for which there are no known causes. In fact, it is estimated that 20% to 30% of all cases of chronic diarrhea have no known cause. Despite numerous tests and analyses, doctors often cannot pinpoint why some patients suffer from chronic diarrhea.

In some cases, doctors may advise patients that the chronic diarrhea problem is caused by stress, emotional problems, psychological problems, and so forth. Accordingly, these doctors may suggest that patients change their lifestyle—by reducing stress, seeking professional counseling, etc.—as a means of coping with the chronic diarrhea. Of course, such treatments can be difficult to implement, expensive, drastic, and ineffective. Thus, such treatments are often disfavored.

Moreover, many of the tests currently used to diagnose chronic diarrhea may require multiple analyses by a doctor. Not only can such a battery of tests require a patient to repeatedly visit the doctor, but more importantly, some of the tests required to diagnose chronic diarrhea may be unreliable as well as uncomfortable, embarrassing, and unpleasant.

Accordingly, it would be an advancement in the art to provide a new method, which may be easily administered and reliable. Moreover, this new method may also provide doctors and researchers with additional information regarding the cause of the chronic diarrhea. Such a method for diagnosing chronic diarrhea is disclosed herein.

### BRIEF SUMMARY

The present invention is directed to methods for diagnosing or obtaining information about chronic diarrhea in humans or any other desired warm-blooded animals. The invention is based upon a non-binding theory that through one or more apocrine mechanisms, a healthy gastrointestinal tract secretes a variety of diverse proteinaceous substances into the intestines, and more specifically, into the colon. Non-limiting examples of proteinaceous substances include mitochondria-containing cell fragments, cytoplasm, mucous, digestive enzymes, and/or antimicrobial proteins. Non-limiting examples of antimicrobial proteins include lysozyme and other polycationic antimicrobials, like histones. Working together or independently, these secretions appear to play a role in the regulation of intestinal flora. For instance, lysozyme, calprotectin, and/or histones have antibacterial characteristics that may help purge the intestines of unhealthy microorganisms. Similarly, the presence of digestive enzymes may indicate a colonic extension or supplementation to normal digestion. Additionally, mitochondria secreted in the gastrointestinal tract may react with oxygen and, thereby, make the gastrointestinal tract an anaerobic environment. Under this theory, chronic diarrhea and other gastrointestinal disorders, such as cancer in the gastrointestinal tract, may be diagnosed by detecting deficient or otherwise abnormal levels of one or more of the aforementioned secretions in the gastrointestinal tract. Similarly, under this theory, chronic diarrhea and other disorders may be treated by regulating or adjusting the abnormal secretion levels.

Accordingly, the described methods may include obtaining a sample of gastrointestinal secretion from a patient. The sample may then be analyzed using one or more conventional or novel techniques. The results of the analysis may then be compared with results obtained from the analysis of control samples, or from samples obtained from individuals who have been diagnosed as having healthy intestines, chronic diarrhea, and/or any other condition or disorder of the gastrointestinal tract. Thus, the control samples may comprise normal and abnormal profiles. Also, if appropriate, the abnormal control samples may be further subset into subcategories of chronic diarrhea or gastrointestinal disorders.

After the patient's results have been compared with the results of the control samples, any abnormal secretion levels in the patient may be determined. One or more abnormal secretion levels may then be correlated with chronic diarrhea or some other form of gastrointestinal disorder. After diagnosis, the patient may be treated for the disorder through regulation or adjustment of the abnormal secretion level. Additionally, the results of the sample obtained from the patient may also be used to provide more diagnostic information about the type or cause of the condition.

### BRIEF DESCRIPTION OF THE SEVERAL VIEWS OF THE DRAWINGS

FIG. 1 is a photograph showing the presence of mitochondria in human colonic lumen, wherein the presence of mitochondria is verified through the use of a mitochondria-specific antibody and a fluorescent stain;

FIG. 2 is a photograph showing the presence of amylase in human colonic lumen, wherein the presence of amylase is verified through the use of an amylase-specific antibody and an immunoperoxidase stain;

FIG. 3 is a photograph showing the presence of amylase in human colonic lumen, wherein the presence of amylase is verified through the use of an amylase-specific antibody and an immunofluorescent stain;

FIG. 4 is a photograph showing the presence of lysozyme in human colonic lumen, wherein the presence of lysozyme is verified through the use of a lysozyme-specific antibody and an immunoperoxidase stain; and

FIG. 5 is a photograph showing the presence of lysozyme in human colonic lumen, wherein the presence of lysozyme is verified through the use of a lysozyme-specific antibody and an immunofluorescent stain.

Together with the following description, the Figures may help demonstrate and explain the principles of the described methods.

### DETAILED DESCRIPTION

The following description supplies specific details in order to provide a thorough understanding. Nevertheless, the skilled artisan would understand that the described methods and any associated techniques can be implemented and used without employing these specific details. Indeed, the described methods can be placed into practice with any modification and can be used in conjunction with any apparatus, systems, components, and/or techniques conventionally used in the industry. For example, while the description below focuses on diagnosing chronic diarrhea in humans, it may also be implemented for any other warm-blooded animal or may be used to diagnose many other gastrointestinal disorders.

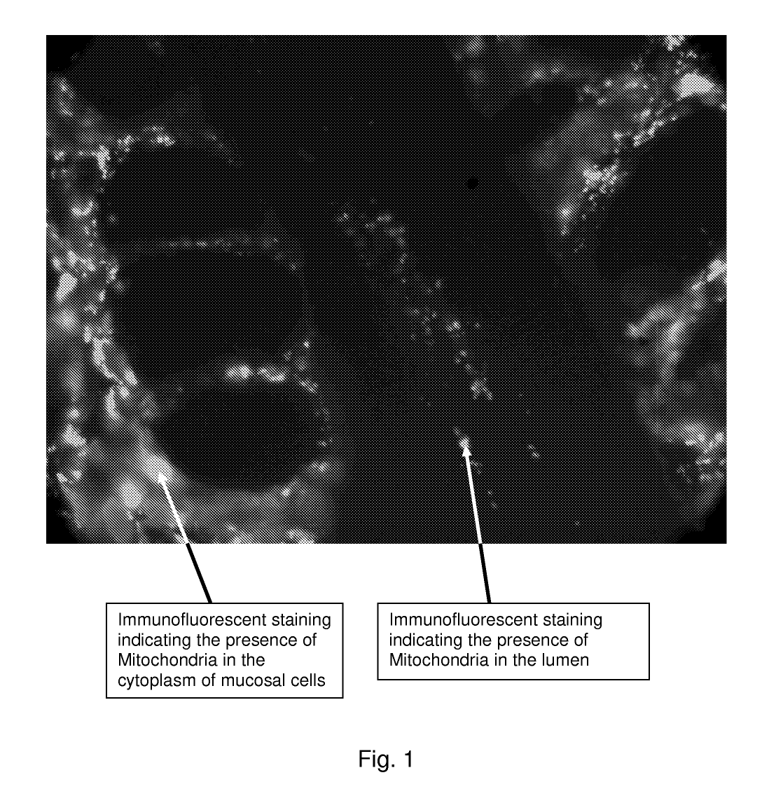

Applicant has discovered that humans and other life forms secrete mitochondria, digestive enzymes, and/or antimicrobial proteins into the gastrointestinal tract. Such secretions may occur in various places throughout the digestive tract and, in particular, may be found in the colon. For example, evidence that mitochondria secretions occur in the gastrointestinal tract may be found in FIG. 1. This Figure contains a picture at 1600× taken using light microscopy for which a sample was prepared using the methods disclosed in U.S. Utility patent application Ser. No. 11/400,468, filed on Apr. 7, 2006, entitled “Compositions and Methods for Preparing Specimens for Microscopic Analysis,” the entire disclosure of which is hereby incorporated by reference. Specifically, with the use of a primary antibody targeted to the ox-phos complex 4, subunit 1 of mitochondria and a fluorescent secondary antibody, FIG. 1 shows the presence of mitochondria in both the cytoplasm of mucosal cells as well as in the lumen, as is indicated by the arrows.

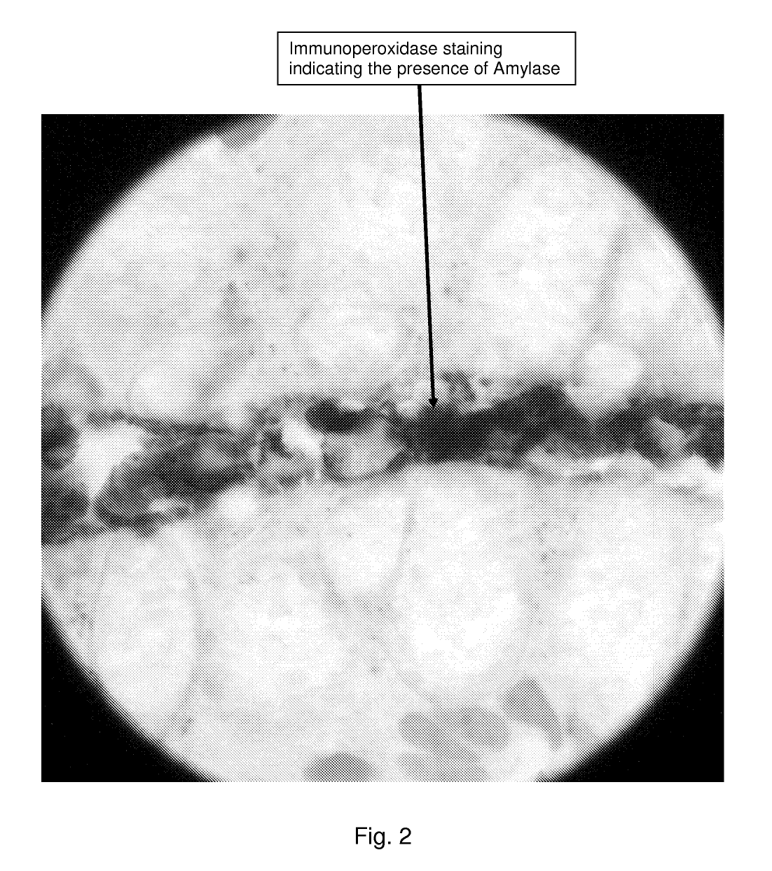

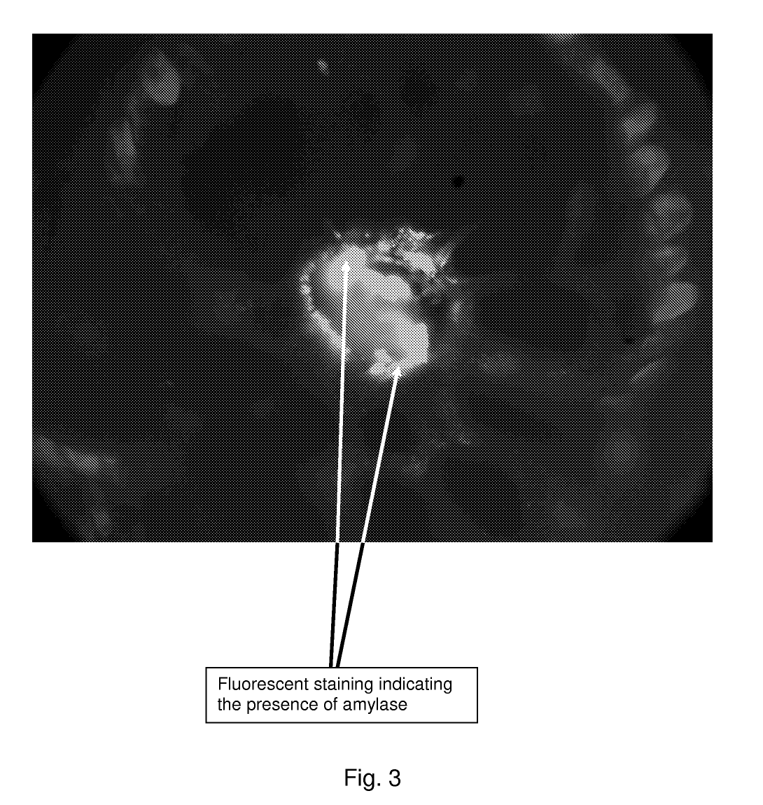

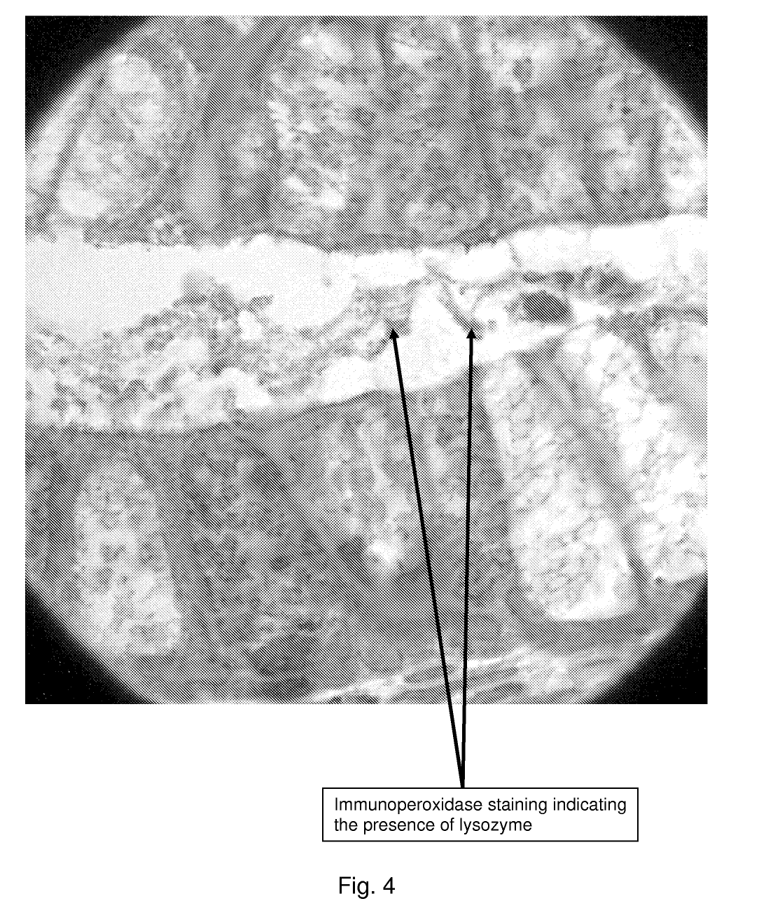

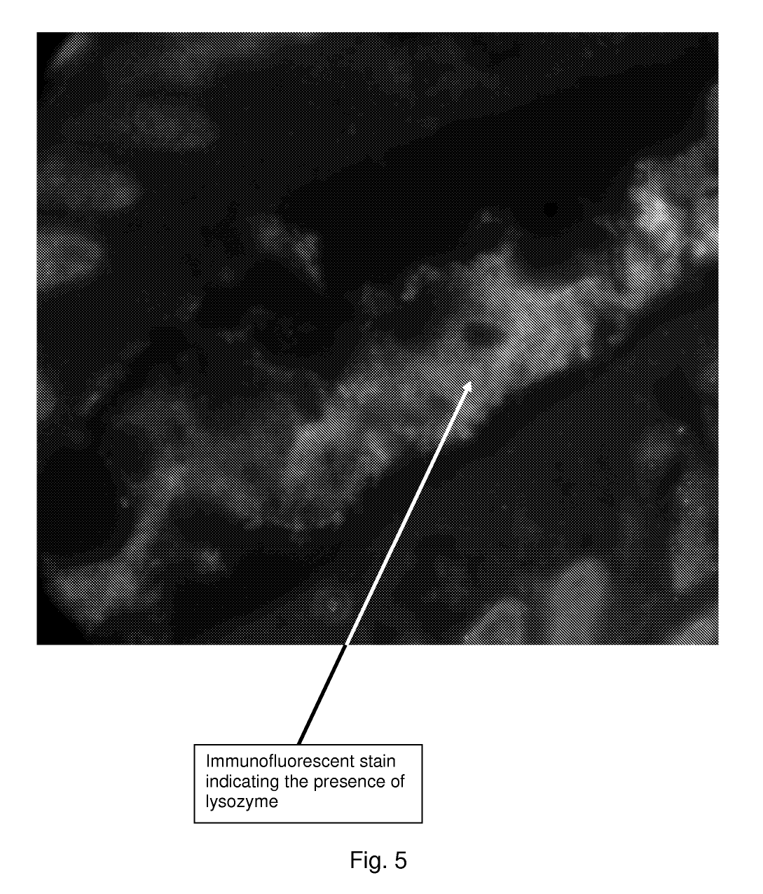

Similarly, FIGS. 2, 3, 4, and 5, which are also pictures taken using light microscopy according to the aforementioned methods, show evidence that digestive enzymes and antimicrobial proteins are secreted into the gastrointestinal tract. For example, FIGS. 2 and 3 show photographs of human colonic lumen that have been treated with a mouse immunoglobulin (IgG) amylase primary antibody, wherein the presence of amylase is indicated with arrows. While the sample in FIG. 2 has been treated with an immunoperoxidase secondary antibody, the sample in FIG. 3 has been treated with an immunofluorescent secondary antibody. Additionally, FIGS. 4 and 5 are photographs using arrows used to indicate the presence of lysozyme in human colonic lumen. While the samples in both FIGS. 4 and 5 have been treated with a rabbit IgG lysozyme primary antibody, the sample in FIG. 4 was treated with an immunoperoxidase secondary antibody, and the sample in FIG. 5 was treated with an immunofluorescent secondary antibody.

The theory that mucus, mitochondrial proteins, digestive enzymes, and/or antimicrobial proteins are secreted and play a prophylactic role in the gastrointestinal tract provides a basis for the described methods. Although the present methods may involve any and/or all of the following steps, in any order, a typical method is described herein. For instance, the described method may comprise collecting samples of gastrointestinal secretions from a control group; or an initial group, or groups, of patients (or humans) who have been diagnosed with healthy gastrointestinal tracts, chronic diarrhea, and/or any other desired form of gastrointestinal disorder. The samples from the control group may be analyzed in order to determine secretion levels and may be used to create a database containing profiles of normal and abnormal gastrointestinal secretions as well as any subset thereof. After such a database is created and specific secretion level abnormalities are identified, patients with these abnormalities may be treated by regulating or adjusting the specific secretion levels. Furthermore, gastrointestinal samples from subsequent patients may be analyzed and compared with the database in order to determine which secretion levels, if any, are abnormal. Once a specific secretion level abnormality has been identified, the patient may be treated. In order to provide a better understanding of the aforementioned method, each step is further discussed below.

As mentioned, the described methods may include collecting samples of gastrointestinal secretions from at least one control group and/or from subsequent patients. The control group in the described methods may be made up of any number of patients with any desired characteristic. For instance, the number of patients in a control group may range anywhere from one single patient to groups of millions of patients, or any number in between. Also, patients in a control group may be of any desired age, sex, weight, height, and the like. Moreover, the patients in a control group may have any known or unknown physical, mental, and/or emotional condition or disorder. For example, patients of a control group may have been previously diagnosed, by any method, as having healthy bowels, chronic diarrhea, and/or another gastrointestinal condition. Some additionally non-limiting examples of gastrointestinal conditions may include Crohn's disease, ulcerative colitis, ulcers, cancer, pre-malignant lesions, malignant lesions, etc. Patients in the control group who have been diagnosed with gastrointestinal disorder may also be subset in any desired manner. For example, patients with chronic diarrhea may be subset into categories or subcategories according to the cause of the diarrhea (e.g., diarrhea caused by disease, parasites, bacteria, viruses, stress, drugs, cancer, etc., or unknown factors).

In some embodiments, a sample may be collected from a patient's colon (i.e., ascending colon, transverse colon, descending colon, and/or sigmoid colon). In other embodiments, a sample of gastrointestinal secretions may be collected from another portion of a patient's gastrointestinal tract, which may range from a patient's mouth to a patient's anus. For example, samples may be collected from a patient's small intestine or stomach.

The collected samples may contain any secretion that may be found in the gastrointestinal tract. For instance, the gastrointestinal samples may contain cytoplasm, mucous, mitochondria, antimicrobial proteins, digestive enzymes, and/or markers for cancer. Some non-limiting examples of antimicrobial proteins may include lysozyme or other polycationic antimicrobials, such as histones. Similarly, some non-limiting examples of digestive enzymes that may be found in the collected samples may include any digestive enzymes found in warm-blooded animals, such as any appropriate form of amylase (e.g., α-amylase, β-amylase, and/or γ-amylase), lipase, peptidase, sucrase, maltase, lactase, isomaltase, eripsin, gelatinase, trypsin, chymotrypsin, etc., and/or combinations thereof.

Samples of gastrointestinal secretions may be collected in any suitable manner. Accordingly, the samples may be collected from stool samples or while the patient is undergoing endoscopy or surgery. For example, samples of gastrointestinal secretions that may contain markers for cancer may be collected from stool samples or directly from pre-malignant and malignant lesions. Indeed, samples of gastrointestinal secretions containing any gastrointestinal secretion may often be obtained as the patient undergoes endoscopy. Because endoscopy is currently one of the more commonly used tests performed on patients with chronic diarrhea, patients may not need to be subjected to additional procedures in order to accomplish the present methods. Furthermore, endoscopy is usually performed on a patient after the patient's bowel and gastrointestinal system have been purged. Accordingly, if gastrointestinal secretions are collected from a patient during endoscopy, these secretions may likely be substantially clean and free of excrement or other impurities. Thus, gastrointestinal secretions collected in this manner may need little purification in order to be used in the present method.

Once a sample of gastrointestinal secretions has been collected, the sample may be analyzed using any known or novel technique in order to determine the presence of one or more specific secretions and/or the level(s) of the specific secretion(s) present in the sample. For instance, some non-limiting examples of methods by which a sample of gastrointestinal secretions may be analyzed may include spectroscopy, chromatography, immunohistochemistry, immunoblotting (e.g., Western blotting), enzyme-linked immunosorbant assays (ELISA), various forms of polymerase chain reaction (PCR), or any other appropriate form of biochemical analysis. Indeed, persons skilled in the art may appreciate that other known and novel analytical methods may be used to analyze the gastrointestinal secretions.

In one example of methods for analysis of samples of gastrointestinal secretions, the samples may be analyzed using spectroscopy and/or chromatography. For instance, in some embodiments, a sample may be analyzed using mass spectroscopy or gas chromatography-mass spectroscopy (GC-MS). In other embodiments, gas chromatography-mass spectroscopy mass spectroscopy (GC-MS/MS) may be used in tandem. In yet other embodiments, surface-enhanced laser desorption ionization time-of-flight (“SELDI-TOF”) or matrix-assisted laser desorption ionization time-of-flight (“MALDI-TOF”) mass spectroscopy may be used to determine the presence and/or levels of particular secretions in a sample.

Where mass spectroscopic analysis is performed, the samples of gastrointestinal secretions may be prepared for analysis through any commonly known method. For instance, some potential methods of sample preparation may be described in an article by David K. Crockett, et al., entitled “Identification of Proteins from Formalin-Fixed Paraffin-Embedded Cells by LC-MS/MS,” the entire disclosure of which is herein incorporated by reference. While the Crockett article relates to recovering tissue specimens from paraffin blocks, it is within the level of skill in the art to prepare samples of gastrointestinal secretions for use with mass spectroscopy, GC-MS/MS, and other analytical methods, through many other methods. For instance, before spectroscopic analysis, a sample of gastrointestinal secretions may be separated into its various components by ultracentrifugation.

As is known in the art, conducting a mass spectrographic analysis on a sample may produce results in the form of a mass chromatogram proteomic pattern with peaks that correspond to the various compounds, proteins, bio-molecules, etc. that may be found in the sample. More specifically, the peaks may include information about the proteome of the sample of gastrointestinal secretions, including information about mitochondria; antimicrobial proteins, such as lysozyme or histones; digestive enzymes, such as amylase, lipase, peptidase, etc.; cytoplasm; cell membranes; mucous; cancer markers; and/or other structures or compounds found in the secretion. Thus, the mass spectroscopy results of the gastrointestinal secretions may include a spectrum or “fingerprint” of expressed genetic information relating to the gastrointestinal tract. Additionally, if the components of the secretions were separated first (e.g., through centrifugation), then separate mass spectroscopy analysis may occur, which may provide a specific proteomic pattern for each component.

In a second example of methods for analyzing samples of gastrointestinal secretions, the sample may be analyzed through any suitable form of immunohistochemistry. For example, monoclonal and/or polyclonal antibodies targeted to one or more specific secretions may be applied to the sample and allowed to complex with corresponding antigens. In this example, a labeled primary antibody could be applied to an antigen (such as lysozyme, histones, amylase, lipase, cancer markers, etc.) in a single stage, or a labeled secondary antibody may be used to target a species-specific part of the structure of a primary antibody. In some instances, the use of a secondary antibody may be advantageous because multiple secondary antibodies may bind to a primary antibody and thereby amplify the signal.

Although any suitable primary and/or secondary antibody(s) may be used to target a desired secretion, some examples of suitable primary antibodies may include anti-histone H2A, anti-histone H2B, anti-histone H3, anti-histone H4, anti-histone H1, anti-lysozyme, anti-amylase, anti-lipase, anti-peptidase, anti-cytochrome oxidase, anti-superoxide dismutase, etc. Moreover, antibodies for any or all mitochondrial proteins may be used as primary antibodies. Some non-limiting examples of antibodies to mitochondrial proteins may include anti-cytochrome C, anti-VDAC/Porin, monoclonal anti-mitochondrial p110, monoclonal anti-TOM22, etc.

As previously alluded to, whether antibodies are used alone, in a single stage, or in conjunction with other antibodies, in multiple stages, the primary and/or secondary antibodies may be directly conjugated with a label. A label may be any chemical group or radioactive atom added to a molecule that is used in order to track material through a reaction or to locate material spatially. In some embodiments, a color or radioactivity may be emitted from the site of an antibody-antigen complex and the emissions may be of varying intensities according to the level of antigen present.

Where immunohistochemistry is implemented for gastrointestinal secretion analysis, any suitable label may be used to help identify desired secretions and/or to determine secretion levels present in the samples. Some examples of suitable labels may include biotin, Texas red, fluorescein, rhodamine, green florescent protein, red florescent protein, cyan florescent protein, yellow florescent protein, horseradish peroxidase, alkaline phosphatase, cyanine, peroxidase, ferratin, phycoerythrin, colloidal gold spheres, gold nano-particles, radiolabels (such as P32, S35, C14, H3, and I125), and the like.

As is known in the art, the results obtained from immunohistochemistry performed on a sample may allow a person or an instrument to analyze the sample and detect the presence of desired secretions and/or the levels of secretions in the sample. The results from such an analysis may be obtained and interpreted in any manner. For example, where the intensity of staining correlates with the amount of protein of interest, a densitometer or absorptiometer may be used to measure protein concentration in a sample. In a second example, a conventional light microscope using bright-field, phase contrast, Nomarski, and/or dark-field microscopy may be used to identify the presence, and to some extent, the level of one or more secretions according the staining of the labels as well as their intensity. In a third example, a microscope with a florescent light (including a confocal microscope) may be used to observe the presence, and to some extent the level, of one or more desired secretions, which can be labeled with a florescent marker, such as fluorescein or rhodamine, via one or more antibodies. In a fourth example, one or more secretions marked with a label, such as ferratin or colloidal gold spheres, may be observed and intensity levels may be noticed through the use of an electron microscope (e.g., transmission, scanning, and/or cryoelectron). In yet a fifth example, one or more secretions marked with a radioactive label may be observed and, to some extent, quantified in a sample through the use of a Geiger counter or film that is sensitive to radioactivity.

In a third example of methods for analyzing samples of gastrointestinal secretions, any suitable form of immunoblotting may be used to determine the presence of specific secretions as well as the levels of one or more specific secretions in the sample. For instance, secretions from a sample may be denatured and then separated through sodium dodecyl sulfate poly-acrylamide gel electrophoresis (SDS/PAGE). After the proteins have been separated, they may be moved from within the gel onto a membrane in order to make the proteins accessible to antibody detection. Such a membrane may be made of nitrocellulose, polyvinylidene difluoride (PVDF), or another appropriate material. After being transferred to the membrane, desired proteins may be probed (detected) using antibodies specific to the target protein, as explained above. For instance, a labeled anti-amylase antibody may be complexed with amylase from the sample of gastrointestinal secretion.

After the primary or secondary antibody produces a signal and any unbound proteins have been washed away, results from sample analysis may be obtained. For instance, size approximations may be taken by comparing stained bands of labeled protein to a marker or ladder loaded during electrophoresis and the amount of target protein may also be indexed to a control. In this manner, the presence of a specific secretion, as well as its level in the sample, may be obtained.

No matter the method of sample analysis, the results of various analyzed samples may be compared to each other and may be used to create a database. Such a database may be created in any desired manner. For example, the results from samples of gastrointestinal secretions of patients diagnosed with various forms of chronic diarrhea or other bowel disorders may be grouped and compared with the results of samples obtained from patients who have been diagnosed as having healthy bowels. For instance, the proteomic patterns obtained through mass spectroscopy of the various groups (e.g., healthy bowels and chronic diarrhea) may be compared and any differences between the group's proteomes may be determined. Similarly, immunohistochemical results from samples obtained from the various groups may be compared and the different staining results (e.g., stain presence and/or intensity) may be determined.

By comparing the results from samples obtained from patients with healthy bowels against the results from samples obtained from patients with various gastrointestinal conditions (e.g., chronic diarrhea, cancer, ulcers, etc.), researchers, doctors, technicians, etc. may be able to identify the secretions present in healthy or normal bowels as well as the normal levels of the various secretions. Such a comparison may also allow researchers, doctors, technicians, etc. to identify the absence or presence of certain secretions as well as levels of particular secretions that are associated with various gastrointestinal conditions.

Such a database may be used for any purpose that aids in the diagnosis of chronic diarrhea or another gastrointestinal disorder. In one example of the utility of such a database, researchers may observe that healthy colons contain amylase and may further identify a normal range for amylase levels in healthy colons. Furthermore, researchers may observe that patients who have been diagnosed with chronic diarrhea lack amylase in the colon or have abnormal levels of amylase (e.g., amylase levels may be above or below the range of amylase levels found in healthy colons). In another similar example, the comparison of the results of samples of gastrointestinal secretions from patients with healthy colons to the results of patients diagnosed with chronic diarrhea may reveal that patients who suffer from chronic diarrhea have abnormal levels of mitochondria, antimicrobial proteins (e.g., lysozyme and/or histones), and/or any desired digestive enzyme, where abnormal refers to levels that are above or below those found in healthy patients. In a non-limiting example, the comparison of digestive enzyme levels from patients diagnosed with healthy bowels and the digestive enzyme levels from patients diagnosed with chronic diarrhea may reveal that a concentration of a digestive enzyme that is 5%, 10%, 20%, etc. above or below the normal levels found in patients with healthy bowels is indicative of chronic diarrhea.

In still another non-limiting example of the utility of such a database, a comparison between the secretions from healthy gastrointestinal tracts and gastrointestinal tracts that have been diagnosed with cancer may reveal the presence of known cancer markers, which may indicate a propensity for or the presence of cancer, in some samples of gastrointestinal tracts from patients diagnosed with cancer. Moreover, such a comparison may also be used to identify additional cancer markers that have been heretofore unidentified. In sum, the characteristics of bowels with various gastrointestinal disorders may be identified and one or more abnormal profiles may be created.

After a database base has been created, samples form subsequent patients may be compared with the database and be used to diagnose gastrointestinal conditions in the subsequent patients. For example, by comparing the proteomic pattern or immunohistochemical staining pattern from a patient's sample to the database, an instrument or individual may be able to identify any abnormalities in the patient's gastrointestinal secretion levels. Also, according to the type of abnormality, the instrument or individual may be able to identify the type gastrointestinal condition the patient has. For instance, an individual may be able to diagnose a patient as having a form of chronic diarrhea associated with a deficiency of amylase in the colon. Thus, by obtaining and analyzing mass spectroscopic proteomic pattern results, or any other form of results, from a particular patient and then comparing the results with those of known profiles in a database, an instrument or individual may be able to diagnose chronic diarrhea and/or another disorder.

Although the comparison of samples may be done in any appropriate manner, adaptive artificial intelligence bioinformatics may be used to read the pathologic states reflected in the proteomic patterns obtained through mass spectroscopy. Bioinformatic pattern recognition tools can recognize complex data from thousands of proteins simultaneously. This information may indicate that the patient has chronic diarrhea or any other gastrointestinal disorder and provide diagnostic information about the type or cause of the condition. Such a method may both speed comparisons as well as increase their accuracy.

As more results are obtained and analyzed, the database may be further subset in any desired manner. Thus, as the database and comparisons reveal more information about gastrointestinal secretions and their roles, the database may also reflect the advanced knowledge and thereby make it easier to diagnose previously un-diagnosable forms of chronic diarrhea and/or other gastrointestinal conditions.

For example, a database may begin simply with a healthy or normal profile and an abnormal or chronic diarrhea profile. As more samples are collected and differences between the samples are identified, each of the categories may be further subset. For instance, the chronic diarrhea category may be split in to several subcategories, such as chronic diarrhea caused by digestive enzyme deficiencies, chronic diarrhea caused by histone deficiencies, chronic diarrhea caused by mitochondria deficiencies, chronic diarrhea caused by cancer, etc. In this example, each of the subcategories could be further subset. For instance, the category of chronic diarrhea caused by digestive enzyme deficiencies may be subset into different categories of specific digestive deficiencies, such as amylase, lipase, or peptidase deficiencies. Additionally, these subcategories of specific digestive deficiencies may also be further subset many subsequent times, according to any desired criteria.

Moreover, as differences are identified between the gastrointestinal secretions of healthy gastrointestinal tracts and the secretions of gastrointestinal tracts with a disorder, like chronic diarrhea, any effective treatments for such conditions may be developed. For example, after ranges of specific secretion levels in healthy colons have been identified, the levels of the specific secretion levels in patients with unhealthy colons may be regulated or adjusted in any suitable manner so as to control, reduce, and/or eliminate the condition. For instance, a patient who is identified as having a form of chronic diarrhea that is associated with a deficiency of amylase in the colon may use dietary supplements, injections, transdermal patches, delayed release enzyme supplements, or suppositories that release amylase in the colon. In yet another example of a method for treating a patient who has been diagnosed with chronic diarrhea, a patient may be prescribed antimicrobials, reducing agents, polycationic compounds, or other supplements alone or with one or more combined in a cocktail depending on their individual secretion composition.

The present invention may be embodied in other specific forms without departing from its methods or other essential characteristics as broadly described herein and claimed hereinafter. The described embodiments and examples are to be considered in all respects only as illustrative, and not as restrictive or limiting. The scope of the invention is, therefore, indicated by the appended claims, rather than by the foregoing description. All changes that come within the meaning and range of equivalency of the claims are to be embraced within their scope.

1. A method for diagnosing a gastrointestinal disorder in a warm-blooded animal comprising the steps of:
obtaining a sample of a gastrointestinal secretion from the warm-blooded animal;
analyzing the sample of gastrointestinal secretion to determine whether levels of digestive enzymes, mitochondria, or antimicrobial proteins in the sample are abnormal; and
correlating an abnormal level of digestive enzymes, mitochondria, or antimicrobial proteins in the sample with the gastrointestinal disorder in the warm-blooded animal from which the sample was obtained.

2. The method of claim 1, wherein the sample of gastrointestinal secretion is obtained from a colon of the warm-blooded animal.

3. The method of claim 2, wherein the gastrointestinal disorder is chronic diarrhea.

4. The method of claim 2, wherein the gastrointestinal disorder is cancer.

5. The method of claim 4, wherein the digestive enzymes comprise amylase, lipase, and peptidase.

6. The method of claim 4, wherein the digestive enzymes comprise amylase.

7. The method of claim 4, wherein the abnormal level comprises a deficiency.

8. The method of claim 4, wherein the method further comprises the step of treating the warm-blooded animal with chronic diarrhea by adjusting or regulating abnormal levels of digestive enzymes, mitochondria, or antimicrobial proteins in the warm-blooded animal with chronic diarrhea.

9. The method of claim 4, wherein the antimicrobial proteins comprise lysozyme or histones.

10. The method of claim 4, wherein the antimicrobial proteins comprise histones.

11. The method of claim 4, wherein the method further comprises a step of comparing results obtained from an analysis performed on the sample of gastrointestinal secretions obtained from the warm-blooded animal with results obtained from analyses performed on a control group in order to determine the differences between each.

12. The method of claim 11, wherein the control group comprises warm-blooded animals diagnosed as having a healthy colon.

13. The method of claim 12, wherein the control group further comprises warm-blooded animals that have been diagnosed with chronic diarrhea or another gastrointestinal disorder.

14. A method for diagnosing chronic diarrhea in a warm-blooded animal comprising the steps of:
obtaining a sample of a gastrointestinal secretion from a colon of the warm-blooded animal;
analyzing the sample of gastrointestinal secretion to determine levels of digestive enzymes present in the sample;
comparing results obtained from the analyzing step with results obtained from a control group in order to determine if the levels of digestive enzymes in the sample are abnormal; and
correlating an abnormal level of digestive enzymes in the sample with chronic diarrhea in the warm-blooded animal from which the sample was obtained.

15. The method of claim 14, wherein the digestive enzymes comprise amylase, peptidase, and lipase.

16. The method of claim 14, wherein the digestive enzymes comprise amylase.

17. The method of claim 14, wherein the abnormal level of digestive enzymes comprises a deficiency.

18. The method of claim 14, wherein the method further comprises a step of treating the warm-blooded animal with chronic diarrhea by adjusting or regulating the abnormal level of digestive enzymes.

19. A method for diagnosing chronic diarrhea in a human comprising the steps of:
obtaining a sample of a gastrointestinal secretion from a colon of the warm-blooded animal;
analyzing the sample of gastrointestinal secretion to determine levels of antimicrobial proteins present in the sample;
comparing results obtained from the analyzing step with results obtained from a control group in order to determine if the levels of the antimicrobial proteins in the sample are abnormal; and
correlating an abnormal level of antimicrobial proteins in the sample with chronic diarrhea in the warm-blooded animal from which the sample was obtained.

20. The method of claim 19, wherein the antimicrobial proteins comprise lysozyme.

21. The method of claim 19, wherein the antimicrobial proteins comprise histones.

22. The method of claim 19, wherein the method further comprises a step of treating the warm-blooded animal with chronic diarrhea by regulating or adjusting the abnormal level of antimicrobial proteins.

23. The method of claim 22, wherein the control group comprises warm-blooded animals diagnosed as having a healthy colon.

24. The method of claim 23, wherein the control group further comprises warm-blooded animals diagnosed as having chronic diarrhea or another gastrointestinal disorder.

25. A method for diagnosing chronic diarrhea in a warm-blooded animal comprising the steps of:
obtaining a sample of a gastrointestinal secretion from a colon of the warm-blooded animal;
analyzing the sample of gastrointestinal secretion to determine levels of mitochondria present in the sample;
comparing results obtained from the analyzing step with results obtained from a control group in order to determine if the levels of mitochondria in the sample are abnormal; and
correlating an abnormal level of mitochondria in the sample with chronic in the warm-blooded animal from which the sample was obtained.
